(19) United States
(12) Patent Application Publication
(10) Pub. No.: US 2021/0170054 A1
(43) Pub. Date: Jun. 10, 2021

(54) NOBLE METAL-COATED MECHANORESPONSIVE VESICLES

(71) Applicants: Andreas Zumbuehl, Bern (CH); BOARD OF REGENTS, THE UNIVERSITY OF TEXAS SYSTEM, Austin, TX (US)

(72) Inventors: Zhenpeng QIN, Richerdson, TX (US); Xiuying LI, Richerdson, TX (US); Frederik NEUHAUS, Fribourg (CH); Andreas ZUMBUEHL, Fribourg (CH); Hejian XIONG, Richardson, TX (US)

(73) Assignee: Board of Regents, the University of Texas System, Austin, TX (US)

(21) Appl. No.: 17/046,385

(22) PCT Filed: Apr. 15, 2019
(86) PCT No.: PCT/US2019/027453

§ 371 Date: Oct. 9, 2020

Related U.S. Application Data
(60) Provisional application No. 62/657,391, filed on Apr. 13, 2018.

(51) Int. Cl. A61K 49/00; A61K 9/00; A61K 9/127; A61K 41/00

(52) U.S. Cl. CPC A61K 49/0084; A61K 9/0019; A61K 49/0041; A61K 41/0028; A61K 9/1271

(57) ABSTRACT

In some aspects, the present disclosure provides mechanosensitive vesicles, which are light sensitive for delivery of a guest molecule. In some embodiments, these vesicles include mechanosensitive phospholipids which have been coated with a noble metal coating.

### PRIORITY CLAIM

This application claims benefit of priority to U.S. Provisional Application Ser. No. 62/657,391, filed Apr. 13, 2018, the entire contents of which are hereby incorporated by reference.

### STATEMENT REGARDING FEDERALLY-SPONSORED RESEARCH OR DEVELOPMENT

This invention was made with government support under Grant No. 1631910 awarded by the National Science Foundation (NSF). The government has certain rights in the invention.

This invention was also made with Swiss government support under Grant No. PP00P2_166209 awarded by the Swiss National Science Foundation (SNSF).

### BACKGROUND

### 1. Field

The present disclosure generally relates to the field of drug delivery. More specifically, it relates to the use of mechanosensitive or mechano-responsive vesicles or liposomes.

### 2. Related Art

Remote modulation of biological activity and behavior is area of importance in the fields of medicine and biology. Light is an important modality to modulate biological systems, such as for photo-induced drug release for treatment of cancer and neurological disorders, modifying cell signaling, and optogenetics. However, light scattering in the tissue significantly limits its tissue penetration to superficial tissue regions (Martelli et al., 2016; Nizamoglu et al., 2016), and often requires the use of invasive optical fibers. Near infrared (NIR) tissue window offers a promising approach to penetrate into deeper tissue regions. Recently, it was demonstrated near-infrared deep brain stimulation via upconversion nanoparticle-mediated optogenetics (Chen et al., 2018). Photo-release of biomolecules in deep tissue regions using NIR stimulation represent a significant challenge since it requires higher light energy exposure and tissue scattering significantly reduces laser energy in deep tissue regions.

Packaging biomolecules inside phospholipid liposomes represent the most important nanoparticle drug delivery system and are already translated into the clinics (Pelaz et al., 2017). In aqueous media, most phospholipids self-assemble into vesicular structures surrounding an aqueous inner cavity (Ramanathan et al., 2013). This compartment can be filled with guest compounds, preventing their exposure to tissue until they reach the target. But as all precision nanomedicines, liposomes show very low targetability to specific tissue regions (Wilhelm et al., 2016). Spatio-temporal addressability of vesicle by an external stimulus would be an important step forward in nanomedicine. Various approaches have been attempted including using temperature sensitive liposomes, which require elevated temperature in the tissue. The inventors have recently presented an ultrafast near-infrared (NIR) light triggered release of bioactive molecules from plasmonic liposomes (Li et al., 2017; Troutman et al., 2009). These liposomes were formulated from standard natural phospholipids and phospholipid mixtures in both the gel and liquid crystalline phase. The release is triggered by ultrashort laser pulses creating nanoscale cavitation bubbles that rapidly burst and transfer mechanical energy to the liposomes with minimal heat dissipation (Lukianova-Hleb et al., 2014). However, it remains a significant challenge to photo-release biomolecules in deep tissue regions due to the significant light scattering and attenuation even for near-infrared light and the high laser energy requirement for photo-release with current compositions. Therefore, there remains a need to develop compositions which have light triggered release of a guest molecule with lower energy.

### SUMMARY

The present disclosure provides mechanosensitive vesicles which are able to release at least a first cargo or guest molecule when exposed to a stress. In some embodiments, the present disclosure provides vesicles comprising:

a) at least one phospholipid type of the formula:

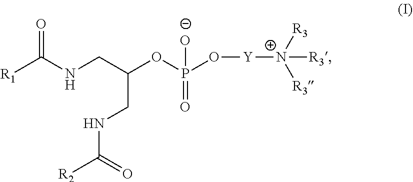

wherein:
R₁ are R₂ are each independently alkyl_{(C≤18)}, alkenyl_{(C≤18)}, alkynyl_{(C≤18)}, or a substituted version of any of these groups;
R₃, R₃′, and R₃″ are each independently hydrogen; or alkyl_{(C≤8)}, alkenyl_{(C≤8)}, alkynyl_{(C≤8)}, acyl_{(C≤8)}, or a substituted version of any of these groups; or
R₃ and R₃′ are taken together and are alkanediyl_{(C≤8)}, alkenediyl_{(C≤8)}, or a substituted version of any of these groups and with the atom to which they are bound form a heterocycloalkyl_{(C≤8)} or a substituted heterocycloalkyl_{(C≤8)}, and R₃″ is hydrogen; or alkyl_{(C≤8)}, alkenyl_{(C≤8)}, alkynyl_{(C≤8)}, acyl_{(C≤8)}, or a substituted version of any of these groups;
Y is alkanediyl_{(C≤8)}, alkenediyl_{(C≤8)}, alkynediyl_{(C≤8)}, or a substituted version of any of these groups; and

b) a noble metal coating;

wherein the noble metal coating is on a surface, inner or outer, of the vesicle. The vesicle may comprise 100% phospholipid, 95%-100% phospholipid, 90%-100% phospholipid, 85%-90% phospholipid, 80%-85% phospholipid or 75%-80% phospholipid, such as the at least one phospholipid type. The remaining vesicle material may be wholly or partially cholesterol, or maybe wholly partially a distinct phospholipid or phospholipids.

In some embodiments, the at least one phospholipid type is further defined as:

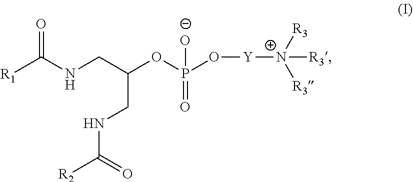

wherein:
R₁ are R₂ are each independently alkyl_{(C≤18)}, alkenyl_{(C≤18)}, alkynyl_{(C≤18)}, or a substituted version of any of these groups;
R₃, R₃′, and R₃″ are each independently hydrogen; or alkyl_{(C≤8)}, alkenyl_{(C≤8)}, alkynyl_{(C≤8)}, acyl_{(C≤8)}, or a substituted version of any of these groups; or
R₃ and R₃′ are taken together and are alkanediyl_{(C≤8)}, alkenediyl_{(C≤8)}, or a substituted version of any of these groups and with the atom to which they are bound form a heterocycloalkyl_{(C≤8)} or a substituted heterocycloalkyl_{(C≤8)}, and R₃″ are each independently hydrogen; or alkyl_{(C≤8)}, alkenyl_{(C≤8)}, alkynyl_{(C≤8)}, acyl_{(C≤8)}, or a substituted version of any of these groups; and
Y is alkanediyl_{(C≤8)} or substituted alkanediyl_{(C≤8)}.

In some embodiments, the at least one phospholipid type is further defined as:

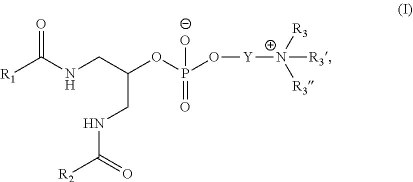

wherein:
R₁ are R₂ are each independently alkyl_{(C≤18)}, alkenyl_{(C≤18)}, alkynyl_{(C≤18)}, or a substituted version of any of these groups;
R₃, R₃′, and R₃″ are each independently hydrogen; or alkyl_{(C≤8)}, alkenyl_{(C≤8)}, alkynyl_{(C≤8)}, acyl_{(C≤8)}, or a substituted version of any of these groups; and
Y is alkanediyl_{(C≤8)} or substituted alkanediyl_{(C≤8)}.

In some embodiments, Y is alkanediyl_{(C≤8)}. In some embodiments, Y is alkanediyl_{(C≥6)} such as ethanediyl. In some embodiments, R₃, R₃′, or R₃″ is alkyl_{(C≤8)} or substituted alkyl_{(C≤8)}. In some embodiments, R₃, R₃′, or R₃″ is alkyl_{(C≤8)}. In some embodiments, R₃, R₃′, or R₃″ is alkyl_{(C≤4)} such as methyl. In some embodiments, R₃, R₃′, and R₃″ are all the same group.

In some embodiments, R₁ or R₂ alkyl_{(C≤18)} or substituted alkyl_{(C≤18)}. In some embodiments, R₁ or R₂ is alkyl_{(C≤18)} such as hexadecanyl. In some embodiments, the phospholipid is further defined as:

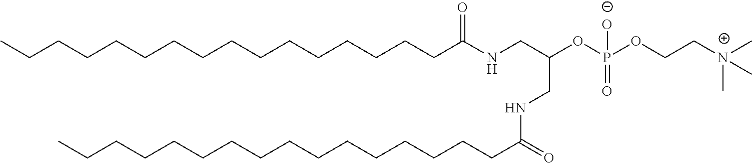 [SMILES: CCCCCCCCCCCCCCCCC(=O)NCC(CNC(=O)CCCCCCCCCCCCCCCC)OP(=O)([O-])OCC[N+](C)(C)C]

In some embodiments, the noble metal coating is a gold coating. In some embodiments, the noble metal coating is a plurality of noble metal nanoparticles. In some embodiments, the vesicles further comprise one or more guest compounds such as a therapeutic agent and/or a diagnostic/visualization agent.

In some aspects, the present disclosure provides pharmaceutical compositions comprising:
a) a vesicle as described herein; and
b) one more excipients.

In some embodiments, the composition is formulated for administration via injection, such as intravenous injection. In some embodiments, the pharmaceutical composition is formulated as a unit dose.

In still yet another aspect, the present disclosure provides methods of delivering one or more guest compounds to a cell comprising:
a) contacting the cell with a vesicle described herein comprising the one or more guest compounds; and
b) irradiating the cell and/or vesicle with an energy source.
   Delivering may be into the bloodstream, through an endoscope, or through a canula. Delivering may be performed prior to activation.

In some embodiments, the energy source is an ultrasound pulse or pulses, or a light pulse or pulses, e.g., a laser pulse, which can be coherent or non-coherent, and may match the noble metal nanoparticles. Ane exemplary laser condition is 28 ps laser; 50 mJ/cm² laser pulse energy. In some embodiments, the laser pulse is a short or an ultrashort laser pulse. In some embodiments, the ultrashort laser pulse has a duration from about 1 ps to about 1 ns or from about 1 ps to about 50 ps. In some embodiments, the laser pulse has a fluence from about 0.1 mJ/cm² to about 500 mJ/cm², from about 1 mJ/cm² to about 500 mJ/cm², from about 1 mJ/cm² to about 100 mJ/cm², from about 1 mJ/cm² to about 75 mJ/cm², from about 50 mJ/cm² to about 100 mJ/cm², or from about 1 mJ/cm² to about 50 mJ/cm². In some embodiments, the laser emits light at a wavelength from about 200 nm to about 1000 nm, from about 600 nm to about 1000 nm, or from about 650 nm to about 800 nm.

In some embodiments, the laser is pulsed more than once. In some embodiments, the laser is pulsed from about 1 time to about 1000 times. In some embodiments, the laser is pulsed from about 1 time to about 50 times. In some embodiments, the one or more guest compounds is a therapeutic agent and/or visualization agent, such as a diagnostic agent. In some embodiments, the therapeutic agent is a signal transduction modulator. In other embodiments, the one or more guest compounds is a dye or a fluorescent dye. In some embodiments, the methods further comprise contacting the vesicle with a high shear environment or a change in shear gradient.

In yet another aspect, the present disclosure provides methods of delivering one or more guest compounds to a cell comprising:
a) contacting the cell with a vesicle described herein comprising the one or more guest compounds; and
b) contacting the vesicle with a high shear environment or a change in shear gradient.
   In some embodiments, the methods further comprise irradiating the cell with an energy source.

In another aspect, the present disclosure provides methods of treating a disease or disorder in patient in need thereof comprising administering to the patient a vesicle, such as a suspension of vesicles, described herein, wherein the vesicle further comprises a therapeutic agent and/or diagnostic agent useful for the treatment and/or diagnosis of the disease or disorder as a guest molecule.

In some embodiments, the methods further comprise irradiating the vesicle with an energy source. In some embodiments, the energy source is a laser. In some embodiments, the laser emits light at a wavelength from about 200 nm to about 1000 nm, from about 600 nm to about 1000 nm, or from about 650 nm to about 800 nm. In some embodiments, the laser is pulsed more than once. In some embodiments, the laser is pulsed from about 1 time to about 1000 times. In some embodiments, the laser is pulsed from about 1 time to about 50 times.

In some aspects, the disclosure relates to the vesicles and compositions of the disclosure for use as a medicament, to the vesicles of the disclosure for delivering a medicament to cells of an individual subject, and to the use of the vesicles of the disclosure in the delivery of a medicament and/or in a formulation of a medicament. In some embodiments, the vesicles of the disclosure are used in combination with exposure of an individual subject to light, preferably infrared light, for example light pulses, more preferably near-infrared light, and most preferably near-infrared laser pulses. In an aspect, the vesicles of the disclosure are used for encapsulating a medicament.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-1D show the design and characterization of mechanosensitive vesicles. FIG. 1A shows the molecular structure of Rad-PC-Rad and DPPC. FIG. 1B shows transmission electron microscopy (TEM) imaging of gold-coated Rad-PC-Rad vesicles. FIG. 1C shows the UV-Vis spectrum of Rad-PC-Rad liposomes with and without gold (Au) coating. FIG. 1D shows the measurement of nanomechanical cavitations generated by near-infrared laser pulse stimulation of gold-coated Rad liposomes.

FIGS. 2A-2C show the mechanical and photo-release of mechanoresponsive Rad-PC-Rad nanovesicles. FIG. 2A shows mechanical stimulation (i.e., vortex shaking) stimulates release from Rad-PC-Rad liposomes and doesn't lead to release from DPPC liposomes.

FIG. 2B shows the comparison of photo-release efficiency of Rad-PC-Rad and DPPC liposomes coated with gold nanoparticles. The arrow shows the reduction in laser pulse energy that leads to approximately 40% release of encapsulated content (404 mJ/cm² for DPPC and 10 mJ/cm² for Rad-PC-Rad). FIG. 2C shows the kinetics of photo-release from gold-coated Rad-PC-Rad and DPPC liposomes.

FIGS. 3A-3C show the photo-release of mechanoresponsive nanovesicles inside living cells in vitro. FIGS. 1A-1B show snapshots and schematic of intracellular release of calcein. Single near-infrared (750 nm) laser pulse is applied at 30 mJ/cm². FIG. 3C shows real-time changes of fluorescent intensity for the photo-stimulated cell.

FIGS. 4A-4D show photo-release of secondary messenger molecules from nanovesicles leads to calcium signaling in vitro. FIG. 4A shows gold-coated nanovesicles are taken up by the cell via endocytosis. Laser stimulation of the nanovesicle leads to intracellular release of a secondary messenger, IP3, which then triggers calcium release inside the cell. FIGS. 4B-4C show snapshots of calcium imaging after photo-release from DPPC (FIG. 4B) and Rad-PC-Rad (FIG. 4C) nanovesicles. FIG. 4D shows quantification of the calcium signal from photo-release using Rad-PC-Rad and DPPC liposomes.

FIGS. 5A-5C show remote photo-release in deep brain regions in vivo. FIG. 5A shows a schematic of experimental procedures. Photosensitive vesicles are injected to different depths in the brain. Then near-infrared light is applied on the brain surface to remotely photo-release the nanovesicles. Afterwards, the brain was removed and frozen to obtain coronal slices for imaging. FIG. 5B shows the comparison of photo-release at different depths in the brain using mechanoresponsive Rad-PC-Rad and standard DPPC liposomes coated with gold particles. 20 pulses of near-infrared laser pulse at 120 mJ/cm² were used. FIG. 5C shows the effect of near-infrared pulse number on the in vivo photo-release of Rad-PC-Rad nanovesicles. Pulse energy density was kept at 120 mJ/cm² and nanovesicles are 2 mm below brain cortical surface.

FIGS. 6A-G show NIR laser pulses-triggered release in brain from mechanosensitive nano-vesicles. (FIG. 6A) Schematic of experimental procedures. Mechanosensitive nano-vesicles were injected to different depths in the brain and then NIR laser pulses were applied on the brain surface. Afterwards, brain was extracted and frozen to obtain coronal slices in order for imaging. (FIG. 6B) Representative calcein fluorescent images of brain sections. Scale bar: 2 mm. (FIG. 6C) Schematic of injection depth versus measured release depth in brain. (FIG. 6D) Box plot of measured release depth in brain. Three brightest sections were chosen for each mouse. (FIG. 6E) Representative calcein fluorescence distribution in each slices for release at different depth (upper: 2 mm, lower: 4 mm). (FIG. 6F) Normalized total calcein fluorescence intensity for each mouse at different depth (upper: 2 mm, lower: 4 mm). (FIG. 6G) Calcein and Texas red fluorescence ratio for each mouse at different depth (upper: 2 mm, lower: 4 mm). Datas were expressed as Mean±SD. *p<0.05,** p<0.01,***p<0.001.

### DETAILED DESCRIPTION OF THE DISCLOSURE

The present disclosure provides gold-coated mechanosensitive or mechano-responsive vesicles or liposomes, which consist of liposomes made from the artificial phospholipid such as Rad-PC-Rad and are already under mechanical stress, as well as pharmaceutical compositions and methods of use thereof. In some embodiments, near-infrared pulses activate the gold-coating to create nanomechanical stress leading to near-complete release of guest compound from the vesicle in sub-seconds. In some embodiments, mechanosensitive vesicles of the present disclosure comprise a gold coating that makes the vesicle sensitive to near-infrared light. Activation of the gold-coated mechanosensitive vesicles with ultrashort laser pulses generates nanoscale cavitation and thus nanomechanical forces. This rapidly and efficiently releases any encapsulated molecules and requires laser energy 40 times lower compared to natural vesicles. Compared to natural phospholipid liposomes, the photo-release is possible at 40 times lower laser energy and tissue penetration is more than doubled. These compositions may be used to trigger the release of biomolecules at deep tissue depth will find broad biomedical applications from cancer treatment to deep brain modulation.

### I. MECHANORESPONSIVE/MECHANOSENSITIVE VESICLES OR LIPOSOMES

In some aspects, the present disclosure provides vesicles comprising a phospholipid of the present disclosure and a noble metal coating, wherein the noble metal coating is on the surface of the vesicle. In some embodiments, the noble metal is gold. In some embodiments, the noble metal coating comprises one or more noble metal nanoparticles dotting the surface of the vesicle. The noble metal nanoparticles may be gold nanoparticles.

Whereas typically vesicles are formed using a flexible and mechanically stable phospholipid bilayer in the liquid crystalline phase, vesicles of the present disclosure comprise 1,3-diamidophospholipids, and optionally other kinds of phospholipids and/or cholesterol, which form stiff, faceted and mechanoresponsive vesicles. These mechanoresponsive vesicles can be induced to release an encapsulated guest compound upon exposure to a physical trigger such as an increase in shear stress or a shear gradient that is for instance found in and around atherosclerotic stenosis. Plasmonic events or ultrasound can generate such or similar physical triggers. In some embodiments, the 1,3-diamidophospholipids include those phospholipids of the formula:

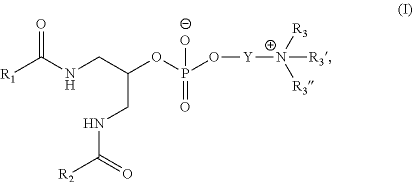

wherein:
R₁ are R₂ are each independently alkyl_{(C≤18)}, alkenyl_{(C≤18)}, alkynyl_{(C≤18)}, or a substituted version of any of these groups;
R₃, R₃′, and R₃″ are each independently hydrogen; or alkyl_{(C≤8)}, alkenyl_{(C≤8)}, alkynyl_{(C≤8)}, acyl_{(C≤8)}, or a substituted version of any of these groups; or
R₃ and R₃′ are taken together and are alkanediyl_{(C≤8)}, alkenediyl_{(C≤8)}, or a substituted version of any of these groups and with the atom to which they are bound form a heterocycloalkyl_{(C≤8)} or a substituted heterocycloalkyl_{(C≤8)};
Y is alkanediyl_{(C≤8)}, alkenediyl_{(C≤8)}, alkynediyl_{(C≤8)}, or a substituted version of any of these groups;
   or a pharmaceutically acceptable salt thereof. The vesicle may comprise 100% phospholipid, 100%-95%-100% phospholipid, 90%-95% phospholipid, 85%-90% phospholipid, 80%-85% phospholipid or 75%-80% phospholipid, such as the at least one phospholipid type. The remaining vesicle material may be wholly or partially cholesterol, or maybe wholly or partially a distinct phospholipid or phospholipids.

### II. ACTIVATION OF NOBLE METAL COATED VESICLES

A. Photo-Induced Activation of Noble Metal Coated Vesicles

The noble metal coating of the mechanosensitive vesicles disclosed herein makes the vesicle sensitivity to near-infrared light. Activation of the gold-coated mechanosensitive vesicles with ultrashort laser pulses generates nanoscale cavitation and thus nanomechanical forces which may be used to trigger the disruption of the vesicle. The generation of these forces cause the release of guest compounds from within the vesicle or the vesicle membrane. In some aspects, the vesicle compositions of the present disclosure are activated upon irradiation with light with a wavelength from about 200 nm to about 1100 nm, such as from about 650 nm to about 900 nm, from about 700 nm to about 800 nm, or from about 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890 to about 900 nm or any range derivable thereof. In some embodiments, the vesicle is activated upon irradiation with light in the near-infrared range such as from 600 nm to about 1100 nm, from about 650 nm to about 800 nm, or from about 700 nm to about 1000 nm.

B. High Shear-Induced Activation of Noble Metal Coated Vesicles

The vesicles of the present disclosure may also be activated by contacting the vesicles with a high shear environment or a shear gradient. In some embodiments, vesicles release their guest compounds upon activation by a mechanical stimulation. In some embodiments, the mechanical stimulation is vortex shaking to create a high shear environment (FIG. 2A). It is contemplated that high shear environments produce in vivo as a result of a disease or disorder may produce a high shear environment and thereby initiate and enhance the release of guest compounds from the vesicles of the present disclosure. Ultrasound may be used to create a high shear environment or shear gradient.

### III. GUEST COMPOUNDS

In an embodiment of the disclosure, a therapeutic agent or agents and/or a diagnostic agent or agents is/are encapsulated within the vesicles or the vesicle membrane of the present disclosure forming a guest compound. The therapeutic agent is an agent capable of treating a disease state or disorder and may be selected from the group consisting of antibiotics, antimicrobials, anticoagulants, antiproliferatives, antineoplastics, antioxidants, endothelial cell growth factors, thrombin inhibitors, immunosuppressants, anti-platelet aggregation agents, collagen synthesis inhibitors, therapeutic antibodies, nitric oxide donors, antisense oligonucleotides, wound healing agents, therapeutic gene transfer constructs, extracellular matrix components, vasodilators, thrombolytics, antimetabolites, growth factor agonists, antimitotics, statins, steroids, steroidal and nonsteroidal anti-inflammatory agents, angiotensin converting enzyme (ACE) inhibitors, free radical scavengers, PPAR-gamma agonists, small interfering RNAs (siRNAs), microRNAs, mRNAs, DNA oligonucleotides, DNA polynucleotides (including those coding for polypeptides) and anti-cancer chemotherapeutic agents. Alternatively, the vesicles may further comprise a diagnostic molecule such as a dye or signaling molecule which is useful to monitor or image a patient.

### IV. PHARMACEUTICAL COMPOSITIONS

In some aspects, the vesicles of the present disclosure will be formulated as pharmaceutical composition, i.e., suitable for administration to patients. Pharmaceutical compositions of the present disclosure comprise an effective amount of a therapeutic agent encapsulated in the vesicle of the present disclosure and dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that contains at least one active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, cryoprotectants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The candidate substance may comprise different types of carriers depending on whether it is to be administered in solid, liquid or aerosol form, and whether it need to be sterile for such routes of administration as injection. The present disclosure can be administered intravenously, intradermally, intraarterially, intraperitoneally, intralesionally, intracranially, intraarticularly, intraprostatically, intrapleurally, intratracheally, intranasally, intravitreally, intravaginally, intrarectally, intralumbally, topically, intratumorally, intramuscularly, subcutaneously, subconjunctival, mucosally, intrapericardially, intraumbilically, intraocularally, orally, locally, via inhalation (e.g., aerosol inhalation), via injection, via infusion, via continuous infusion, via localized perfusion bathing target cells directly, via a catheter, via a lavage, in creams, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference). Additionally, the pharmaceutical composition may be injected directly into the tissue of the deep brain. Alternatively, the pharmaceutical composition may be injected directly into a tumor.

The actual dosage amount of a composition of the present disclosure administered to an animal patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The candidate substance may be formulated into a composition in a free base, neutral or salt form. Pharmaceutically acceptable salts include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

In other embodiments, one may use eye drops, nasal solutions or sprays, aerosols or inhalants in the present disclosure. Such compositions are generally designed to be compatible with the target tissue type. In a non-limiting example, nasal solutions are usually aqueous solutions designed to be administered to the nasal passages in drops or sprays. Nasal solutions are prepared so that they are similar in many respects to nasal secretions, so that normal ciliary action is maintained. Thus, in particular embodiments the aqueous nasal solutions usually are isotonic or slightly buffered to maintain a pH of about 5.5 to about 6.5. In addition, antimicrobial preservatives, similar to those used in ophthalmic preparations, drugs, or appropriate drug stabilizers, if required, may be included in the formulation. For example, various commercial nasal preparations are known and include drugs such as antibiotics or antihistamines.

In certain embodiments the candidate substance is prepared for administration by such routes as oral ingestion. In these embodiments, the solid composition may comprise, for example, solutions, suspensions, emulsions, tablets, pills, capsules (e.g., hard- or soft-shelled gelatin capsules), sustained release formulations, buccal compositions, troches, elixirs, suspensions, syrups, wafers, or combinations thereof. Oral compositions may be incorporated directly with the food of the diet. Preferred carriers for oral administration comprise inert diluents, assimilable edible carriers or combinations thereof. In other aspects of the disclosure, the oral composition may be prepared as a syrup or elixir. A syrup or elixir, and may comprise, for example, at least one active agent, a sweetening agent, a preservative, a flavoring agent, a dye, a preservative, or combinations thereof.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

In particular embodiments, prolonged absorption of an injectable composition can be brought about by the use in the compositions of agents delaying absorption, such as, for example, aluminum monostearate, gelatin or combinations thereof.

The skilled artisan is directed to “Remington's Pharmaceutical Sciences” 15th Edition, chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

### V. DEFINITIONS

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means=NH; “cyano” means —CN; “isocyanate” means —N═C═O; “azido” means —N₃; in a monovalent context “phosphate” means —OP(O)(OH)₂ or a deprotonated form thereof; in a divalent context “phosphate” means —OP(O)(OH)O— or a deprotonated form thereof; “mercapto” means —SH; and “thio” means ═S; “sulfonyl” means —S(O)₂—; and “sulfinyl” means —S(O)—.

In the context of chemical formulas, the symbol “—” means a single bond, “═” means a double bond, and “≡” means triple bond. The symbol “ [symbol] ” represents an optional bond, which if present is either single or double. The symbol “ [symbol] ” represents a single bond or a double bond. Thus, the formula

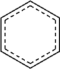

covers, for example,

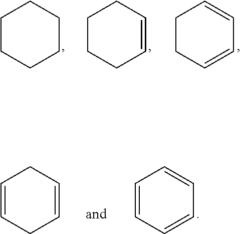

And it is understood that no one such ring atom forms part of more than one double bond. Furthermore, it is noted that the covalent bond symbol “—”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.,

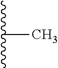

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a variable is depicted as a “floating group” on a ring system, for example, the group “R” in the formula:

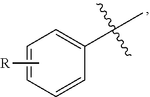

then the variable may replace any hydrogen atom attached to any of the ring atoms, including a depicted, implied, or expressly defined hydrogen, so long as a stable structure is formed. When a variable is depicted as a “floating group” on a fused ring system, as for example the group “R” in the formula:

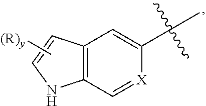

then the variable may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the R enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” defines the exact number (n) of carbon atoms in the group/class. “C≤n” defines the maximum number (n) of carbon atoms that can be in the group/class, with the minimum number as small as possible for the group/class in question. For example, it is understood that the minimum number of carbon atoms in the groups “alkyl_{(C≤8)}”, “cycloalkanediyl_{(C≤8)}”, “heteroaryl_{(C≤8)}”, and “acyl_{(C≤8)}” is one, the minimum number of carbon atoms in the groups “alkenyl(c)”, “alkynyl_{(C≤8)}”, and “heterocycloalkyl_{(C≤8)}” is two, the minimum number of carbon atoms in the group “cycloalkyl_{(C≤8)}” is three, and the minimum number of carbon atoms in the groups “aryl_{(C≤8)}” and “arenediyl_{(C≤8)}” is six. “Cn-n′” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C5 olefin”, “C5-olefin”, “olefin_{(C5)}”, and “olefin_{C5}” are all synonymous. When any of the chemical groups or compound classes defined herein is modified by the term “substituted”, any carbon atom in the moiety replacing the hydrogen atom is not counted. Thus methoxyhexyl, which has a total of seven carbon atoms, is an example of a substituted alkyl_{(C1-6)}. Unless specified otherwise, any chemical group or compound class listed in a claim set without a carbon atom limit has a carbon atom limit of less than or equal to twelve.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” signifies that the compound or chemical group so modified is an acyclic or cyclic, but non-aromatic compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” signifies that the compound or chemical group so modified has a planar unsaturated ring of atoms with 4n+2 electrons in a fully conjugated cyclic π system.

The term “alkyl” when used without the “substituted” modifier refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or Bu), and —CH₂C(CH₃)₃ (neo-pentyl) are non-limiting examples of alkyl groups. The term “alkanediyl” when used without the “substituted” modifier refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. The term “alkylidene” when used without the “substituted” modifier refers to the divalent group ═CRR′ in which R and R′ are independently hydrogen or alkyl. Non-limiting examples of alkylidene groups include: ═CH₂, ═CH(CH₂CH₃), and ═C(CH₃)₂. An “alkane” refers to the class of compounds having the formula H—R, wherein R is alkyl as this term is defined above. When any of these terms is used with the “substituted” modifier, one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups.

The term “alkenyl” when used without the “substituted” modifier refers to a monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” when used without the “substituted” modifier refers to a divalent unsaturated aliphatic group, with two carbon atoms as points of attachment, a linear or branched, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H—R, wherein R is alkenyl as this term is defined above. Similarly, the terms “terminal alkene” and “α-olefin” are synonymous and refer to an alkene having just one carbon-carbon double bond, wherein that bond is part of a vinyl group at an end of the molecule. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂₀H, or —S(O)₂NH₂. The groups —CH═CHF, —CH═CHCl and —CH═CHBr are non-limiting examples of substituted alkenyl groups.

The term “alkynyl” when used without the “substituted” modifier refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —C≡CH, —C≡CCH₃, and —CH₂C≡CCH₃ are non-limiting examples of alkynyl groups. The term “alkynediyl” when used without the “substituted” modifier refers to a divalent unsaturated aliphatic group, with two carbon atoms as points of attachment, a linear or branched, a linear or branched acyclic structure, no carbon-carbon double bond, at least one carbon-carbon triple bonds, and no atoms other than carbon and hydrogen. The groups —C≡C—, —C≡CCH₂-, and —CH₂CH≡CHCH₂— are non-limiting examples of alkenediyl groups. An “alkyne” refers to the class of compounds having the formula H—R, wherein R is alkynyl. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂₀H, or —S(O)₂NH₂.

The term “aryl” when used without the “substituted” modifier refers to a monovalent unsaturated aromatic group with an aromatic carbon atom as the point of attachment, said carbon atom forming part of a one or more aromatic ring structures, each with six ring atoms that are all carbon, and wherein the group consists of no atoms other than carbon and hydrogen. If more than one ring is present, the rings may be fused or unfused. Unfused rings are connected with a covalent bond. As used herein, the term aryl does not preclude the presence of one or more alkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl (e.g., 4-phenylphenyl). The term “arenediyl” when used without the “substituted” modifier refers to a divalent aromatic group with two aromatic carbon atoms as points of attachment, said carbon atoms forming part of one or more six-membered aromatic ring structures, each with six ring atoms that are all carbon, and wherein the divalent group consists of no atoms other than carbon and hydrogen. As used herein, the term arenediyl does not preclude the presence of one or more alkyl groups (carbon number limitation permitting) attached to the first aromatic ring or any additional aromatic ring present. If more than one ring is present, the rings may be fused or unfused. Unfused rings are connected with a covalent bond. Non-limiting examples of arenediyl groups include:

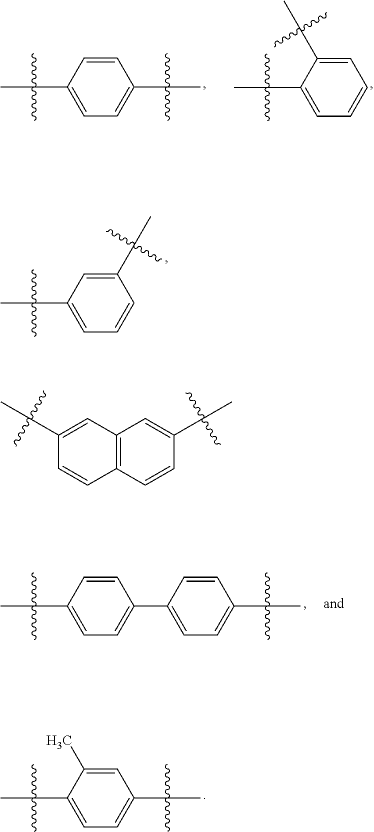

An “arene” refers to the class of compounds having the formula H—R, wherein R is aryl as that term is defined above. Benzene and toluene are non-limiting examples of arenes. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂₀H, or —S(O)₂NH₂.

The term “heterocycloalkyl” when used without the “substituted” modifier refers to a monovalent non-aromatic group with a carbon atom or nitrogen atom as the point of attachment, said carbon atom or nitrogen atom forming part of one or more non-aromatic ring structures, each with three to eight ring atoms, wherein at least one of the ring atoms of the non-aromatic ring structure(s) is nitrogen, oxygen or sulfur, and wherein the heterocycloalkyl group consists of no atoms other than carbon, hydrogen, nitrogen, oxygen and sulfur. If more than one ring is present, the rings are fused. As used herein, the term does not preclude the presence of one or more alkyl groups (carbon number limitation permitting) attached to one or more ring atoms. Also, the term does not preclude the presence of one or more double bonds in the ring or ring system, provided that the resulting group remains non-aromatic. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, pyranyl, oxiranyl, and oxetanyl. The term “N-heterocycloalkyl” refers to a heterocycloalkyl group with a nitrogen atom as the point of attachment. N-pyrrolidinyl is an example of such a group. When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “acyl” when used without the “substituted” modifier refers to the group —C(O)R, in which R is a hydrogen, alkyl, cycloalkyl, or aryl as those terms are defined above. The groups, —CHO, —C(O)CH₃ (acetyl, Ac), —C(O)CH₂CH₃, —C(O)CH(CH₃)₂, —C(O)CH(CH₂)₂, —C(O)C₆H₅, and —C(O)C₆H₄CH₃ are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkyl group, as defined above, attached to a —CHO group. When any of these terms are used with the “substituted” modifier one or more hydrogen atom (including a hydrogen atom directly attached to the carbon atom of the carbonyl or thiocarbonyl group, if any) has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups.

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects. Alternatively, the term “about” refers to the stated value, plus or minus 5% of that stated value.

The term “coating” as used herein refers to a layer of material or compounds that is located on the surface of the vesicle but need not be completely covering the vesicle or entirely on the surface of the vesicle.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result.

An “isomer” of a first compound is a separate compound in which each molecule contains the same constituent atoms as the first compound, but where the configuration of those atoms in three dimensions differs.

As used herein, the term “noble metal” refers to the group of elements selected from the group consisting of gold, silver, and copper and the platinum group metals (PGM) platinum, palladium, osmium, iridium, ruthenium and rhodium. In certain particular embodiments of the present disclosure, the noble metal is selected from the group consisting of gold, silver, and copper. In some particular embodiments, the noble metal is gold or silver.

As used herein, the term “nanoparticle” refers to an association of 2-1000 atoms of a metal. Nanoparticles may have diameters in the range of about 1 to about 100 nm. In some embodiments, the nanoparticles have a diameter in the range of about 10 nm to about 100 nm. In other particular embodiments, the nanoparticles comprise approximately 2-1000, approximately 2-500, approximately 2-250, approximately 2-100, approximately 2-25 atoms, or approximately 2-10 atoms. As used herein, the terms “nanoparticle composition” references to a noble metal nanoparticle as described herein.

As used herein, the term “patient” or “subject” refers to a living animal organism, such as a human, monkey, cow, sheep, goat, dog, cat, mouse, rat, guinea pig, or transgenic species thereof. In certain embodiments, the patient or subject is a mammal. In some embodiments, the patient is a human. Non-limiting examples of human patients are adults, juveniles, infants and fetuses.

“Pharmaceutically acceptable salts” means salts of compounds of the present disclosure which are pharmaceutically acceptable, as defined above, and which possess the desired pharmacological activity. Non-limiting examples of such salts include acid addition salts formed with inorganic acids such as hydrochloric acid, hydrobromic acid, sulfuric acid, nitric acid, and phosphoric acid; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, and trimethylacetic acid. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Non-limiting examples of acceptable organic bases include ethanolamine, diethanolamine, triethanolamine, tromethamine, and N-methylglucamine. It should be recognized that the particular anion or cation forming a part of any salt of this disclosure is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

The above definitions supersede any conflicting definition in any of the reference that is incorporated by reference herein. The fact that certain terms are defined, however, should not be considered as indicative that any term that is undefined is indefinite. Rather, all terms used are believed to describe the disclosure in terms such that one of ordinary skill can appreciate the scope and practice the present disclosure.

### VI. EXAMPLES

The following examples are included to demonstrate particular embodiments of the disclosure. It should be appreciated by those of skill in the art that the techniques disclosed in the examples which follow represent techniques discovered by the inventor to function well in the practice of the disclosure, and thus can be considered to constitute particular modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Discussion and General Strategy

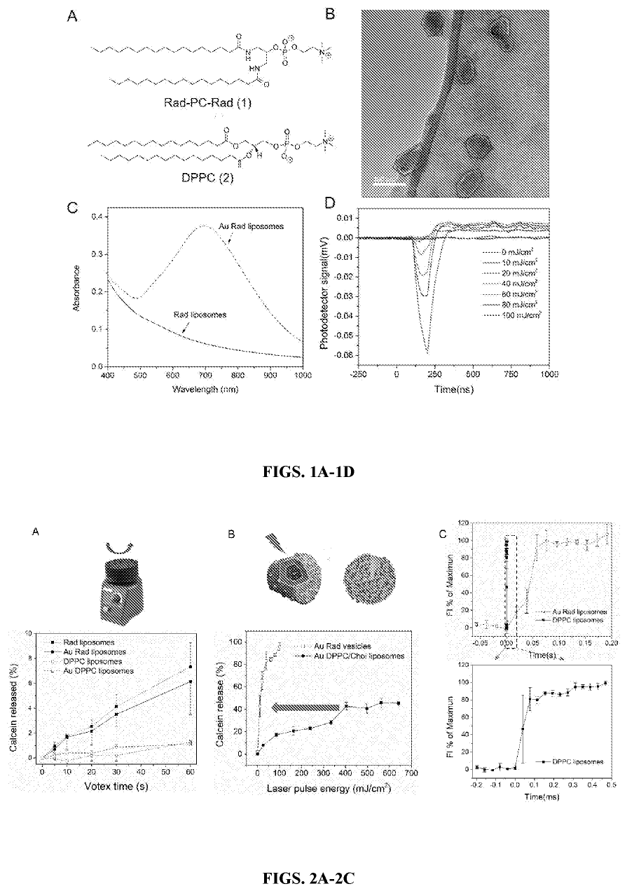

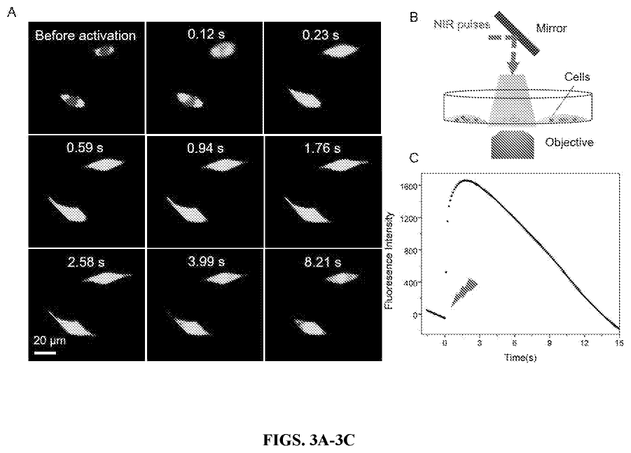

First, plasmonic mechanosensitive liposomes were prepared and characterized. Rad-PC-Rad was synthesized as previously reported (Neuhaus et al., 2018 and Fedotenko et al., 2010). Phosphorus oxytrichloride was substituted with 1,3-dichloropropanol and Boc-protected ethanolamine to yield a reasonably stable phosphoramidate. Transforming this intermediate via a diazide, followed by a reduction led to the diamine. The heptadecanoyl chains were accessible via heptadecanoic acid chloride. Coupling diamine and heptadecanoic acid chloride yielded the headgroup-protected phosphoramidate. Acidic headgroup-deprotection and quaternization with dimethyl sulfate yielded the final 1,3-diamidophospholipid Rad-PC-Rad (FIG. 1A). Using Rad-PC-Rad, gold coated vesicles were prepared with near-infrared peak plasmon resonance in the near-infrared window (700-900 nm, FIGS. 1B and 1C). The Rad-PC-Rad vesicles showed highly faceted geometries in cryo-TEM images (FIG. 1B). Similar to gold-coated DPPC liposomes, gold-coated Rad-PC-Rad vesicles created plasmonic nanobubbles upon picosecond (ps) pulsed laser activation (FIG. 1D). The results suggested that gold-coated Rad-PC-Rad vesicles can be activated by near-infrared pulsed laser.

Next, the photo-release of mechanosensitive liposomes was evaluated. As synthesized, the Rad-PC-Rad vesicles release their cargo induced by a mechanical stimulation by vortex shaking to create a high shear environment (FIG. 2A) (Holme et al., 2012 and Neuhaus et al., 2018). In contrast, the standard DPPC vesicles do not respond to the increased shear stress during vortex shaking. With ultrashort laser pulse irradiation (28 ps, 740 nm), both DPPC and Rad-PC-Rad nanovesicles show release (FIG. 2B). However, quantitative comparison of the two vesicles shows that the Rad-PC-Rad releases 40% of its encapsulated biomolecules at 40 times lower laser fluence compared with DPPC (404 mJ/cm² vs 10 mJ/cm²). Further comparison of the fast release kinetics shows that the Rad-PC-Rad vesicle releases its content over a period of 0.1 s, compared with the 0.1 ms for DPPC vesicle (FIG. 2C). Comparison of the release efficiency and kinetics shows a striking difference between the mechanosensitive vesicle and DPPC. The release efficiency measurement shows an “ON/OFF” behavior, i.e., nearly complete release above the release threshold while zero release below the threshold with single laser pulse. On the other hand, the DPPC shows an opposite behavior, with the release efficiency gradually increasing with laser energy but never reaching complete release with single laser pulse. This could be due to the fact that the nanomechanical stress attenuates small membrane packing defects, destroying the integrity of the Rad-PC-Rad vesicles that is already under internal stress and slow to recover once perturbed. On the other hand, for DPPC vesicles, the nanomechanical stress (or nanocavitations) simply deforms DPPC vesicles to release small amounts of encapsulated biomolecules and quickly recovers afterwards. This theory is also in agreement with the time-lapsed measurement of release kinetics (FIG. 2C).

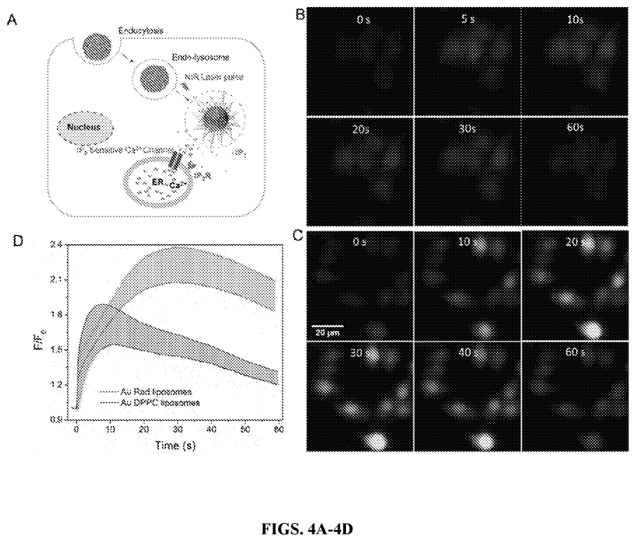

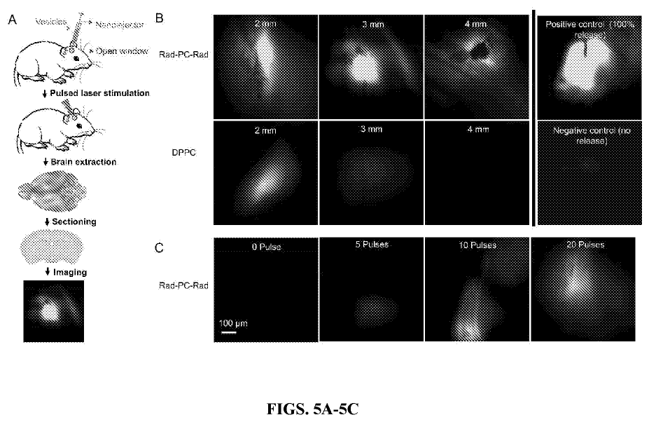

Next, the in vitro photo-release of mechanosensitive liposomes was investigated. The photo-release of fluorescent dye (calcein) inside the cells was first tested. Calcein-loaded mechanosensitive liposomes were allowed to be taken up by the cell through endocytosis. Once in the endosomes, laser stimulation of the gold-coated Rad-PC-Rad nanovesicles triggers the release of calcein into the cell cytosol (FIGS. 3A-3C). This demonstrates that the nanomechanical stimulation by laser pulse not only triggers the release of the molecules from the nanovesicles, but also facilitates its release into the cell cytosol. Next, using an intracellular calcium-signaling pathway, the cellular response of photo-releasing Rad-PC-Rad and DPPC nanovesicles was investigated (FIGS. 4A-4D). Specifically, intracellular IP3 release leads to calcium (Ca²⁺) release from intracellular calcium storage such as nucleus and endoplasmic reticulum. Comparing the calcium responses using Rad-PC-Rad and DPPC nanovesicles, photo-release of inositol trisphosphate (IP₃) from Rad-PC-Rad vesicles leads to a more prolonged and higher calcium response than DPPC vesicles. This indicates a higher release efficiency from Rad-PC-Rad vesicles, which takes the cell longer to restore the calcium into intracellular compartments (Clapham, 2007).

Next, the use of the mechanosensitive liposomes for in vivo biomolecule release was tested. Gold-coated liposomes were injected at different depths in the brain of live C57BL/6J mice and released using laser irradiation from the brain surface (FIG. 5A). Release of calcein from the liposomes leads to green fluorescence, which is otherwise self-quenched (at a high concentration, 75 mM) inside liposomes. The results show a clear release from gold-coated DPPC liposomes at 2 mm depth from the brain surface. For gold-coated Rad-PC-Rad vesicles, however, clear release is observable down to 4 mm (FIG. 5B). As a further comparison, increasing the number of laser pulses leads to more efficient dye release (FIG. 5C). This demonstrates that the photo-release from mechanosensitive Rad-PC-Rad vesicles have potential to treat diseases in deeper tissue regions and modulating deep brain activity. It is contemplated that using the photo-release in deep tissue regions to manipulate brain activity, for instance in different behavioral tests including Pavlovian fear conditioning may deliver positive results.

### Example 2—Synthetic Methods, Charaterization, and Data Related to the Delivery of Guest Compounds to Cells

A. Preparation and Characterization of Mechanosensitive Liposomes

Mechanosensitive liposomes were prepared by a two-step method. First, naked Rad-PC-Rad vesicles were prepared following a previously reported method (Neuhaus et al., 2017). To begin, 10 mg of the lipid was dissolved in CHCl₃ in a 25 mL glass bottom flask. After evaporation of the organic solvent, the film was further dried under high vacuum (40 mbar) overnight. The film was then hydrated with 10 mM phosphate buffered saline (PBS) under 65° C. for 30 min. Next, at least 5 freeze-thaw cycles (liquid N₂ to 65° C.) were carried out before the suspension was extruded through 200 and 100 nm polycarbonate membranes (Whatman, USA) 11 times using a Mini Extruder (Avanti Polar Lipids, USA). Free calcein or IP₃ was removed by size-exclusion chromatography with Sephacryl S-1000 column. Second, gold nanoparticles were deposited onto the liposome surface following a previously reported method with minor modification (Troutman et al., 2009). Gold chloride solution was added (10 mM) and gently mixed with the liposome suspension (1.5 mM lipid concentration) in a molar ratio of 1:4 until uniformly distributed, followed by the addition of ascorbic acid solution (40 mM) with the same volume. Following reduction, a plasmonic liposomes sample was dialyzed against 10 mM PBS under room temperature for 2 h to remove unreacted gold chloride and ascorbic acid.

The sizes of mechanosensitive liposomes and uncoated liposomes were determined by dynamic light scattering measurement (Malvern ZetaSizer Nano ZS). Extinction spectrum of mechanosensitive liposomes in PBS were taken with a spectrophotometer (DU800, Beckman Coulter). The morphology of the plasmonic liposomes was observed by atransmission electron microscope (TEM, JEOL-1400+) at an accelerating voltage of 150 keV. A droplet of mechanosensitive liposomes at a lipid concentration of 100 μM was placed on a carbon support film, and the excess liquid was evaporated under room temperature for 1 h before imaging. Cryo-TEM was also performed to image the gold-coated nanovesicles.

The plasmonic nanobubbles were measured with an optical pump-probe technique. Mechanosensitive liposomes were placed on a glass slice covered by a cover slice and irradiated with laser pulses with different fluence (0, 10, 20, 40, 60, 80, 100 mJ/cm²) Mechanosensitive liposomes absorb the near-infrared excitation laser pulse (i.e., pump, 740 nm) and create nanobubbles which strongly scatters another continuous laser beam (i.e., probe, 633 nm), leading to a decrease in the transmitted laser intensity. The axial intensity of the beam was recorded with a fast photodetector (FPD510-FV, Thorlabs) which was displayed by a digital oscilloscope (LeCroy WaveRunner204Xi-A) and analyzed as a time-response.

B. In Vitro Release

Mechanical force triggered release was studied following a literature method (Holme et al., 2012) with minor modification. To begin, the purified liposomal suspensions were diluted with PBS 10 times and vortexed for a discrete amount of time (0, 5, 10, 20 and 60 s) at 2,500 rpm. Release of calcein was measured by a plate reader (Synergy 2, Bio-Tek) wavelengths of 485 nm (excitation) and 535 nm (emission). Liposomal samples treated by 1% Triton-X100 was use as a control for maximum cargo release. Calcein release was calculated by the following equation.

% Release=(F−Fᵢ)/(Fₜₒₜₐᵢ−Fᵢ)×100% (1)

where F represents fluorescence after laser irradiation, Fᵢ represents initial fluorescence, Fₜₒₜₐₗ represents fluorescence with 100% release induced by Triton-X treatment.

The release kinetics of mechanosensitive liposomes were investigated following a literature method (Holme et al., 2012). To begin, an aliquot of well-dispersed calcein-loaded mechanosensitive liposomes was placed on a glass slide, covered by a cover slide, and sealed by nail polish. The samples were then immobilized onto a microscope (Olympus IX73) stage and irradiated with a single laser pulse (laser beam size: around 100 μm, wavelength: 740 nm). A high speed digital camera (Hamamatsu Photonics, ORCA-Flash 4.0) was used to record the real-time fluorescent intensity profile. A series of fluorescent images were obtained, and the fluorescence intensity was analyzed by Image J.

A capillary flow model was used to test the laser energy dependent relase. To begin, mechanosensitive liposomes were flowed through a capillary with inner diameter 150 μL. The flow rate was calculated and controlled by a low flow peristaltic pump (Cole Parmer) to ensure each mechanosensitive liposome was exposed to single laser pulse. Laser pulses with different energy (0, 10, 20, 40, 60, 80, 100 mJ/cm²) were tested. Liposomal suspension after irradiation were collected at the end of the capillary and the fluorescence intensity were measure by a plate reader. Calcein release percentage was calculated following the equation (1) described above.

C. Intracellular Calcein and IP3 Release

To monitor intracellular release of calcein from mechanosensitive liposomes, Raw 264.7 cells were seeded and cultured in 25 mm glass bottom dishes in DMEM medium supplemented with 10% fetal bovine serum for 24 h. The cells were washed with PBS and replaced with fresh medium that contains calcein loaded mechanosensitive liposomes. After 3 h incubation, cell nuclei were stained with 5 μg/mL Hoechst 33342 for 5 min. Cells were then washed with PBS for 3 times and supplied with fresh DMEM medium prior to laser exposure (740 nm, single pulse, 30 mJ/cm²). To study the intracellular distribution of mechanosensitive liposomes, late endosomes and lysosomes were stained with LysoTracker Red DND-99 for 30 min after endocytosis.

To test the capability of intracellular release of biological active compound upon near infrared laser pulse irradiation, a secondary messenger IP₃, which regulates cell calcium signaling, was encapsulated into mechanosensitive liposomes. Raw 264.7 cells in 25 mm glass bottom dishes were incubated with IP₃ loaded mechanosensitive liposomes (with and without gold coating) for 2 h. After incubation, mechanosensitive liposomes containing cell medium were discarded and washed with PBS for 3 times. To observe changes in calcium concentration, cells were loaded with a calcium indicator (1 μM Fluo-4, 30 min). The fluorescence intensity of Fluo-4 increases 100 folds upon binding to Ca²⁺. After washed with PBS for 3 times, cell medium was replaced with fresh medium and imaged under fluorescence microscope (Olympus IX73). Single near-infrared pulse (740 nm, single pulse, 30 mJ/cm²) was used to activate mechanosensitive liposomes. Image sequences of Raw 264.7 cells before and after laser activation were recorded and the fluorescence intensity of Fluo-4 was analyzed as a function of time.

D. In vivo release

The in vivo light triggered release was tested in the brain tissue of C57BL/6 mice. To begin, the mice were first anesthetized by 2-3% isoflurane and then a window with size around 4-5 mm was opened in the skull by a drill. The window was washed with artificial cerebrospinal fluid (aCSF) or PBS at least 3 times to remove any bone residue or blood. Calcein loaded liposomal suspensions (1 μL, 75 mM) were injected into brain tissues with defined depth by a nanoinjection through the open window. Dextran-Texas red (MW 70 KDa, 1 mg/mL in PBS) was co-injected with liposomal suspension to localized liposomes upon application. After injection, laser beam (740 nm, 120 mJ/cm², 100 μm in diameter) was scanned across the surface of the window. Scan speed, beam diameter and pulse repetition rate were synchronized in order to provide different pulses exposure. After laser stimulation, mice were sacrificed and the brain tissue were collected, embedded and frozen at −20° C. Frozen brain sections (20 m) were obtained using a cryostat and then visualized using a confocal microscope using a 0× objective.

E. Materials

1,3-diheptadecanamidopropan-2-yl (2-(trimethylammonio)ethyl)phosphate (Rad-PC-Rad) was provided by the Andreas Zumbuehl Laboratory. Dipalmitoylphosphatidylcholine (DPPC) and cholesterol were purchased from Avanti Polar Lipids, Incorporated. L-ascorbic acid was purchased from Thermo Fisher Scientific. Calcein sodium salt was purchased from Alfa Aesar. Dextran-Texas red (MW 70 KDa) and gold chloride was purchased from Sigma-Aldrich.

F. Methods

Preparation of Au-Rad-lip (gold-coated Rad-PC-Rad liposome). Briefly, 5 mg of the lipid were dissolved in CHCl₃ in a 25 mL glass bottom flask. After evaporation of the organic solvent, the film was further dried under high vacuum (40 mbar) overnight. Then the film was hydrated with 75 mM calcein in 10 mM phosphate buffered saline (PBS) under 65° C. for 30 min. Then at least 5 freeze-thaw cycles (liquid N₂ to 65° C.) were carried out before the suspension was extruded through 200 and 100 nm polycarbonate membranes (Whatman, USA) for 11 times using a Mini Extruder (Avanti Polar Lipids, USA). Free calcein was removed by size-exclusion chromatography with Sephacryl S-1000 column. Second, gold nanoparticles were decorated onto the liposome surface following a previous reported method with minor modification. Gold chloride solution was added (10 mM) and gently mixed with liposome suspension (0.75 mM lipid concentration) in a molar ratio of 1:2 until uniformly distributed, followed by the addition of ascorbic acid solution (40 mM) with the same volume. Following reduction, Au-Rad-lip was dialyzed against 10 mM PBS under room temperature for 2 h to removed unreacted gold chloride and ascorbic acid. As a standard control group, gold-coated DPPC liposomes (Au-DPPC-lip) were prepared as similar methods.

Calcein release in brain. The in vivo light triggered release was tested in the brain tissue of C57BL/6 mice. Briefly, the mice were firstly anesthetized by 2-3% isoflurane and then a window with size around 3 mm was opened in the skull by a drill. The window was washed with artificial cerebrospinal fluid (ACSF) for at least 3 times to remove any bone residue or blood. A needle with a tip diameter of 0.5 mm was inserted into right visual cortex and targeted to the coordinate of (0.14 mm anterior, 2 mm lateral) (relative to bregma). Calcein loaded Au-Rad-lip or Au-DPPC-lip (1 μL) were injected into brain tissues with defined depth (1 mm, 2 mm and 4 mm) through the open window. Dextran-Texas red (MW 70 KDa, 1 mg/mL in PBS) was co-injected with liposomal suspension to localized liposomes upon application. A pump was used to control the infusion flow rate as 0.1 L/min. After injection, laser beam (740 nm, 170 mJ/cm², 150 μm in diameter) was scanned across the surface of the window. Scanning pattern was set as a circle with diameter of 3 mm and step size of 150 μm. Scan speed, beam diameter and pulse repetition rate were synchronized in order to provide 20 pulses exposure. After laser stimulation, mice were sacrificed, and the brain tissue were collected and frozen at −20° C. Frozen brain sections (40 μm) were obtained using a cryostat from the front to back of brain. Calcein and Texas red fluorescence in the sections were detected by Olympus VS120 100—Slide Scanning System with a 2× objective. Fluorescence intensity was quantitively analyzed by Image J. Two-sample t-test in Origin 9.1 software was conducted for statistical analysis.

G. Results and Discussion

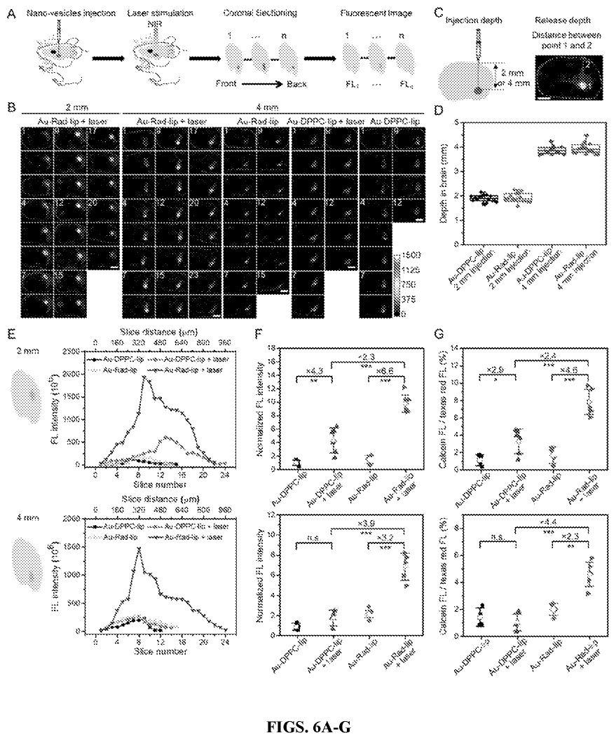

NIR laser pulses-triggered release from mechanosensitive nanovesicles was demonstrated at the different depths in the mouse brain (FIG. 6A, 1 mm, 2 mm and 4 mm). Since deeper tissue penetration is of interest, so the inventors focused on 2 mm and 4 mm depths. Release of calcein from the nano-vesicles leads to green fluorescence, which is otherwise self-quenched (at a high concentration, 75 mM) inside liposomes. From the calcein fluorescent images (FIG. 6B), higher intensity with larger area was observed for Au-Rad-lip group both at 2 mm and 4 mm depth. The actual calcein release depth in brain was measured and matched well with injection depth (FIGS. 6C-D). Both average and total calcein fluorescence intensity were quantitively analyzed on each section and plotted versus slice number. As for release at 2 mm depth, the fluorescence distribution showed intensity increase with laser stimulation, while there was a sharper and larger peak for Au-Rad-lip compared with a smaller peak for Au-DPPC-lip (FIG. 6E). After summing up the fluorescence intensity for each mouse, the inventors observed that calcein fluorescence was 2.3-fold higher in Au-Rad-lip group than that in Au-DPPC-lip group under laser irradiation (FIG. 6F). Moreover, the fluorescence distribution was almost the same for Au-DPPC-lip with or without laser at 4 mm depth, while the higher fluorescent signal for Au-Rad-lip group with laser indicates light-triggered photorelease (FIGS. 6E-F). Calcein fluorescence was 3.9-fold higher in Au-Rad-lip group than that in Au-DPPC-lip group. The fluorescence ratio between calcein and Texas red showed similar results, where Texas red dye is co-injected into the brain with the nanovesicles (FIG. 6G). These results demonstrate that the photo-release was more efficient from mechanosenstive nano-vesicles in brain and can be observable down to 4 mm. This technique provides significant potential to treat diseases in deeper tissue regions and modulating deep brain activity.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and/or methods and in the steps or in the sequence of steps of the method described herein without departing from the concept, spirit and scope of the disclosure. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VII. REFERENCES

The following references, to the extent that they provide exemplary procedural or other details supplementary to those set forth herein, are specifically incorporated herein by reference:
Braun et al., ACS Nano, 3(7):2007-2015, 2009.
Chen et al., Science, 359(6376):679-684, 2018.
Clapham et al., Cell, 131(6):1047-1058, 2007.
Fedotenko et al., Tetrahedron Lett., 51:5382-5384, 2010.
Holme et al., Nat. Nanotechnol., 7:536-543, 2012.
Li et al., Adv. Funct. Mater., 27:1605778, 2017.
Lukianova-Hleb et al., Nat. Med., 20:778-784, 2014.
Martelli et al., Sci. Rep., 6:27057, 2016.
Mellal et al., J. Mater. Chem., 2:247-252, 2014.
Neuhaus et al., Angew. Chem. Int. Ed., 56:6515-6518, 2017.
Neuhaus et al., Langmuir, 34:3215-3220, 2018.
Nizamoglu et al., Nat. Comm., 7(10374):1-7, 2016.
Pelaz et al., ACSNano, 11:2313-2381, 2017.
Ramanathan et al., Phys. Chem. Chem. Phys., 15:10580-10611, 2013.
Troutman et al., Adv. Mater., 21:2334-2338, 2009.
Wilhelm et al., Nat. Rev. Mater., 1:1-12, 2016.

1. A vesicle comprising:
a) at least one phospholipid type of the formula: 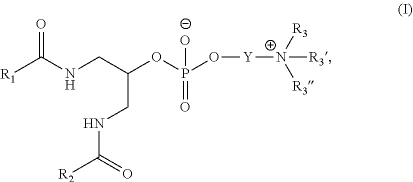
wherein:
R₁ are R₂ are each independently alkyl_{(C≤18)}, alkenyl_{(C≤18)}, alkynyl_{(C≤18)}, or a substituted version of any of these groups;
R₃, R₃′, and R₃″ are each independently hydrogen; or
alkyl_{(C≤8)}, alkenyl_{(C≤8)}, alkynyl_{(C≤8)}, acyl_{(C≤8)}, or a substituted version of any of these groups; or
R₃ and R₃′ are taken together and are alkanediyl_{(C≤8)}, alkenediyl_{(C≤8)}, or a substituted version of any of these groups and with the atom to which they are bound form a heterocycloalkyl_{(C≤8)} or a substituted heterocycloalkyl_{(C≤8)}, and R₃″ is hydrogen; or alkyl_{(C≤8)}, alkenyl_{(C≤8)}, alkynyl_{(C≤8)}, acyl_{(C≤8)}, or a substituted version of any of these groups;
Y is alkanediyl_{(C≤8)}, alkenediyl_{(C≤8)}, alkynediyl_{(C≤8)}, or a substituted version of any of these groups; and
b) a noble metal coating;
wherein the noble metal coating is on the surface of the vesicle.

2. The vesicle of claim 1, wherein the at least one phospholipid type is further defined as: 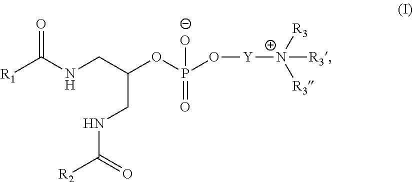
wherein:
R₁ are R₂ are each independently alkyl_{(C≤8)}, alkenyl_{(C≤18)}, alkynyl_{(C≤18)}, or a substituted version of any of these groups;
R₃, R₃′, and R₃″ are each independently hydrogen; or
alkyl_{(C≤8)}, alkenyl_{(C≤8)}, alkynyl_{(C≤8)}, acyl_{(C≤8)}, or a substituted version of any of these groups; or
R₃ and R₃′ are taken together and are alkanediyl_{(C≤8)}, alkenediyl_{(C≤8)}, or a substituted version of any of these groups and with the atom to which they are bound form a heterocycloalkyl_{(C≤8)} or a substituted heterocycloalkyl_{(C≤8)}, and R₃″ are each independently hydrogen; or alkyl_{(C≤8)}, alkenyl_{(C≤8)}, alkynyl_{(C≤8)}, acyl_{(C≤8)}, or a substituted version of any of these groups; and
Y is alkanediyl_{(C≤8)} or substituted alkanediyl_{(C≤8)}.

3. The vesicle according to claim 1, wherein the at least one phospholipid type is further defined as: 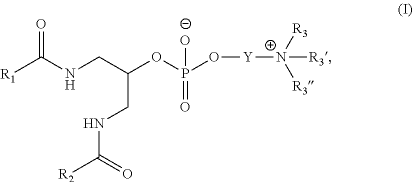
wherein:
R₁ are R₂ are each independently alkyl_{(C≤18)}, alkenyl_{(C≤18)}, alkynyl_{(C≤18)}, or a substituted version of any of these groups;
R₃, R₃′, and R₃″ are each independently hydrogen; or
alkyl_{(C≤8)}, alkenyl_{(C≤8)}, alkynyl_{(C≤8)}, acyl_{(C≤8)}, or a substituted version of any of these groups; and
Y is alkanediyl_{(C≤8)} or substituted alkanediyl_{(C≤8)}.

4. The vesicle according to claim 1, wherein Y is alkanediyl_{(C≤8)}.

5. The vesicle according to claim 1, wherein Y is alkanediyl_{(C≤6)}.

6. The vesicle according to claim 1, wherein Y is ethanediyl.

7. The vesicle according to claim 1, wherein R₃, R₃′, or R₃″ is alkyl_{(C≤8)} or substituted alkyl_{(C≤8)}.

8. The vesicle according to claim 1, wherein R₃, R₃′, or R₃″ is alkyl_{(C≤8)}.

9. The vesicle according to claim 1, wherein R₃, R₃′, or R₃″ is alkyl_{(C≤4)}.

10. The vesicle according to claim 1, wherein R₃, R₃′, or R₃″ is methyl.

11. The vesicle according to claim 1, wherein R₃, R₃′, and R₃″ are all the same group.

12. The vesicle according to claim 1, wherein R₁ or R₂ alkyl_{(C≤18)} or substituted alkyl_{(C≤18)}.

13. The vesicle according to claim 1, wherein R₁ or R₂ is alkyl_{(C≤18)}.

14. The vesicle according to claim 1, wherein R₁ or R₂ is hexadecanyl.

15. The vesicle according to claim 1, wherein the phospholipid is further defined as:

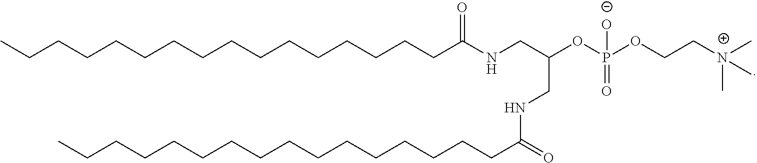 [SMILES: CCCCCCCCCCCCCCCCC(=O)NCC(CNC(=O)CCCCCCCCCCCCCCCC)OP(=O)([O-])OCC[N+](C)(C)C]

16. The vesicle according to claim 1, wherein the noble metal coating is a gold coating.

17. The vesicle of claim 16, wherein the noble metal coating is a plurality of noble metal nanoparticles.

18. The vesicle according to claim 1, further comprising a guest compound.

19. The vesicle of claim 18, wherein the guest compound is a therapeutic agent.

20. A pharmaceutical composition comprising:
a) a vesicle according to claim 1; and
b) at least one excipient.

21. The pharmaceutical composition of claim 20, wherein the composition is formulated for administration via injection.

22. The pharmaceutical composition of claim 20, wherein the pharmaceutical composition is formulated as a unit dose.

23. A method of delivering one or more guest compounds to a cell comprising:
a) contacting the cell with a vesicle according to claim 1 comprising the one or more guest compounds; and
b) irradiating the cell with an energy source.

24. The method of claim 23, wherein the energy source is a laser pulse or an ultrasound pulse.

25. The method of claim 23, wherein the laser pulse is an ultrashort laser pulse.

26. The method of claim 25, wherein the ultrashort laser pulse has a duration from about 1 ps to about 1 ns.

27. The method of claim 25, wherein the ultrashort laser pulse has a duration from about 1 ps to about 50 ps.

28. The method according to claim 24, wherein the laser pulse has a fluence from about 0.1 mJ/cm² to about 500 mJ/cm².

29. The method of claim 28, wherein the fluence is from about 1 mJ/cm² to about 500 mJ/cm².

30. The method of claim 29, wherein the fluence is from about 1 mJ/cm² to about 100 mJ/cm².

31. The method of claim 30, wherein the fluence is from about 1 mJ/cm² to about 75 mJ/cm².

32. The method of claim 30, wherein the fluence is from about 50 mJ/cm² to about 100 mJ/cm².

33. The method of claim 31, wherein the fluence is from about 1 mJ/cm² to about 50 mJ/cm².

34. The method according to claim 24, wherein the laser emits light at a wavelength from about 200 nm to about 1000 nm.

35. The method of claim 34, wherein the wavelength is from about 600 nm to about 1000 nm.

36. The method of claim 35, wherein the wavelength is from about 650 nm to about 800 nm.

37. The method according to claim 24, wherein the laser is pulsed more than once.

38. The method according to claim 24, wherein the laser is pulsed from about 1 time to about 1000 times.

39. The method according to claim 23, wherein the laser is pulsed from about 1 time to about 50 times.

40. The method according to claim 23, wherein the guest compound is a therapeutic agent.

41. The method according to claim 23, wherein the therapeutic agent is a signal transduction modulator.

42. The method according to claim 23, wherein the guest compound is a diagnostic reagent, such as a fluorescent dye.

43. The method according to claim 23, further comprising contacting the vesicle with a high shear environment.

44. A method of delivering one or more guest compounds to a cell comprising:
a) contacting the cell with a vesicle according to claim 1 comprising the one or more guest compounds; and
b) contacting the vesicle with a high shear environment.

45. The method of claim 44, further comprising irradiating the cell with an energy source, such as a laser pulse or an ultrasound pulse.

46. A method of treating a disease or disorder in patient in need thereof comprising administering to the patient a vesicle according to claim 1, wherein the vesicle further comprises a therapeutic agent and/or a visualization agent useful for the treatment and/or diagnosis of the disease or disorder as a guest molecule.

47. The method of claim 46, wherein the method further comprises irradiating the vesicle with an energy source.

48. The method of claim 47, wherein the energy source is a laser.

49. The method according to claim 26, wherein the laser emits light at a wavelength from about 200 nm to about 1000 nm.

50. The method of claim 49, wherein the wavelength is from about 600 nm to about 1000 nm.

51. The method of claim 50, wherein the wavelength is from about 650 nm to about 800 nm.

52. The method according to claim 46, wherein the laser is pulsed more than once.

53. The method according to claim 46, wherein the laser is pulsed from about 1 time to about 1000 times.

54. The method of claim 53, wherein the laser is pulsed from about 1 time to about 50 times.
